(19) United States
(12) Patent Application Publication
(10) Pub. No.: US 2021/0282430 A1
(43) Pub. Date: Sep. 16, 2021

(54) ANIMAL FEED AND METHODS TO PROVIDE SUCH FEED

(71) Applicant: Nutreco Nederland B.V., Boxmeer (NL)

(72) Inventors: Petra Johanna Roubos-van den Hil, Boxmeer (NL); Theodorus A.T.G. Van Kempen, Venray (NL)

(21) Appl. No.: 16/326,436

(22) PCT Filed: Aug. 25, 2017
(86) PCT No.: PCT/NL2017/050559

§ 371 Date: Feb. 19, 2019

(30) Foreign Application Priority Data
Aug. 26, 2016 (NL) 2017375

(51) Int. Cl. A23K 10/30; A23K 20/105; A23K 50/30; A23K 50/60; A23K 50/75; A61K 9/00; A61K 36/07

(52) U.S. Cl. CPC A23K 10/30; A23K 20/105; A23K 50/30; A61K 36/07; A23K 50/75; A61K 9/0056; A23K 50/60

(57) ABSTRACT

A new animal feed comprising Agaricus blazei Murill (ABM) mycelium and one or more C₁-C₁₆ organic acids is disclosed. The disclosure also pertains to a composition comprising Agaricus blazei Murill (ABM) mycelium and one or more C₁-C₁₆ organic acids, to a corresponding kit-of-parts, to a method to feed an animal by providing feed to the animal comprising Agaricus blazei Murill mycelium and one or more C₁-C₁₆ organic acids, and to a method to provide animal feed by mixing Agaricus blazei Murill mycelium and one or more C₁-C₁₆ organic acids with protein and/or carbohydrates and/or fats to provide the feed.

### CROSS-REFERENCE TO RELATED APPLICATIONS

This application is a national phase entry under 35 U.S.C. § 371 of International Patent Application PCT/NL2017/050559, filed Aug. 25, 2017, designating the United States of America and published in English as International Patent Publication WO 2018/038615 A1 on Mar. 1, 2018, which claims the benefit under Article 8 of the Patent Cooperation Treaty to The Netherlands Patent Application Serial No. 2017375, filed Aug. 26, 2016.

### TECHNICAL FIELD

This disclosure pertains to animal feed in general.

### BACKGROUND

Animal feed is used to support the normal metabolism of animals in order for these animals to stay healthy and grow to their full capabilities. In particular, for food-producing animals, it is very important that the feed optimally supports their health since this is often reflected in their performance as measured by establishing the average daily weight gain of an animal, its weight at the age of slaughter or its age at the slaughter weight. In particular, in raising food-producing animals, much attention is given to controlling the spread of bacteria within a group of animals kept at a particular production site. In particular, since such bacteria might be pathogenic to the animal itself (infection thus reducing the animal's health status and hence its performance) or possibly also to consumers of the animal (humans or other animals). Common methods to reduce the spread of bacteria are the use of antibiotics, and/or to vaccinate the animal. Another method used is containment (quarantine) of the animal in combination with sterilizing its feed. This method, however, is not suitable for raising animals for consumption purposes because of the high costs involved.

### BRIEF SUMMARY

Provided is an animal feed that is particularly suitable to improve animal wellbeing, preferably to reduce or mitigate infection with ubiquitous pathogenic bacteria such as bacteria belonging to the enterobacteriaceae, in particular, Salmonella species and Escherichia species.

An animal feed has been devised comprising Agaricus blazei Murill (ABM) mycelium and one or more C₁-C₁₆ organic acids. Agaricus blazei Murill is also called Agaricus blazei brasiliensis, Agaricus subrufescens, or Agaricus rufotegulis. At present, it is thought that Agaricus subrufescens is the correct name; however, in this application, the more common name Agaricus blazei Murill will be used. Hereinbelow, the abbreviation ABM and the terms Agaricus blazei Murill are used interchangeably.

Surprisingly, it was found that when feeding an animal with feed that comprises mycelium of ABM and one or more C₁-C₁₆ organic acids, i.e., an organic compound with acidic properties (see Nomenclature of Organic Chemistry: IUPAC Recommendations and Preferred Names 2013, Author(s) Henri A Favre, Warren H Powell, Chapter P-1: P-10 Introduction, for the definition of an organic compound), in particular, a hydrocarbon having at least one carboxylic group, as well as the organic compounds' salts and esters thereof (since it is known that both these forms are able to release the actual organic acid in the feed material), general health status and hence growth performance of the animal can be improved significantly. In particular, it was found that the colonisation with pathogenic bacteria belonging to the group of the enterobacteriacea is possibly reduced, showing as less bacteria being present in the animal's faeces. This inherently means that the spreading of the bacteria to other animals is also reduced. The reason for this is not 100% clear. It is noted that the use of mycelium of Agaricus blazei Murill for administration to laying hens is known from WO2013/171194. It is described that the presence of the ABM mycelium in the feed improves the egg laying and optionally the egg shell quality and the egg laying period. In the art it is also known to use C₁-C₁₆ organic acids as feed preservatives, i.e., to reduce microbial growth in the feed itself (during stocking the feed). Little is known about the effect of these acids on the growth of pathogens after a host animal gets infected, let alone the combined effect when using the mycelium of ABM in the feed at the same time (either separately or mixed into the same feed composition).

It is noted that the mycelium of ABM and the organic acid(s) do not need to be present in the same feed material at the same time. It is essential that the various feed components (solid feed, drinking water etc.) taken by an animal as a whole comprise both ingredients, such that at least in the gastro-intestinal tract both ingredients are combined and act in accordance with this disclosure.

As feed preservatives, typically fatty acids are being used, that is, any acid comprising a hydrocarbon chain and at least one terminal carboxylic group. In particular, small chain C₁-C₇ acids such as formic acid, propionic acid, lactic acid, citric acid, fumaric acid, benzoic acid and sorbic acid are commonly applied, but also C₇-C₁₆ medium chain fatty acids such as caprylic acid, capric acid, lauric acid and palmitic acid are used. The same acids can be applied in the current disclosure, either alone or in a mixture incorporating various short chain and/or medium chain acids.

The disclosure also pertains to a composition comprising Agaricus blazei Murill (ABM) mycelium and one or more C₁-C₁₆ organic acids. Such a composition can be used to be mixed with nutritional ingredients to provide an animal feed in line with the disclosure or, for example, fed separately, for example, mixed with drinking water (separate from the actual feed), such that it is ingested by an animal and is united with the feed in the gastro intestinal tract of the animal. The disclosure also pertains to a kit-of-parts comprising a first constituting part that comprises Agaricus blazei Murill (ABM) mycelium and a second constituting part comprising one or more C₁-C₁₆ organic acids, and optionally an instruction to orally administer both these parts of the kit to an animal. It is noted that for the sense of the disclosure, the parts do not need to be present in one single container. It is foreseen that the parts are provided in separate containers, not packed together, but with the clear intention (for example, by indications provided on a web-site, separate leaflet, etc.) to be used according to the teaching of the disclosure, for example, by adding one or both parts to animal feed, and/or one or both parts to the drinking water that is offered to the animal in conjunction with its feed.

The disclosure also enables a method to feed an animal by providing feed to the animal comprising Agaricus blazei Murill mycelium and one or more C₁-C₁₆ organic acids. This can be accomplished, for example, by having both ingredients present in the animal feed, or by feeding the animal with a first substance comprising the mycelium of ABM and a second substance comprising the organic acids (for example, drinking water in which the acids are present). The disclosure also enables a method to provide animal feed by mixing Agaricus blazei Murill mycelium and one or more C₁-C₁₆ organic acids with protein and/or carbohydrates and/or fats to provide the feed.

### Definitions

Animal feed is a composition comprising animal nutrients such as fats and/or proteins and/or carbohydrates that is fed to an animal to provide in its metabolic requirements. Animal feed can be a nutritional complete feed (i.e., providing all required nutrients to support a normal metabolism of the animal), but it may also be a premix or other composition that contains only part of the required nutrients, to be mixed with other nutrients or fed separately from these other nutrients.

The total daily intake of feed is the complete mass of feed an animal takes per day, excluding drinking water.

### Embodiments

In a first embodiment of the feed according to the disclosure, the ABM mycelium is present in an amount of 0.01 to 10 kg per ton of total daily intake of feed. In other words, the total amount of feed (excluding the drinking water) as is fed to the animal comprises per 1000 kilograms, 0.01 to 10 kg of mycelium of ABM. This amount can be present in a nutritional complete feed as such, at a level of 0.01 to 10 kg per ton of that feed material, or may, for example, be present in a concentrated feed material (exceeding 10 kg/ton feed material) as long as the amount per total daily intake of feed is between 0.01 and 10 kg ABM mycelium per ton. In particular, the ABM mycelium is fed at an amount of 0.05 to 2 kg per ton of total daily intake of feed. These amounts appear to suffice for use according to the disclosure.

In a further embodiment, the one or more C₁-C₁₆ acids are present in an amount of 0.1 to 10 kg per ton of total daily intake of feed, in particular, in an amount of 0.5 to 6 kg per ton of total daily intake of feed.

In yet another embodiment, the ABM mycelium is grown on a grain substrate, in particular, a rye (Secale cereal) or millet (Panicum miliaceum) substrate. In a further embodiment, the grain substrate with the mycelium grown thereon is incorporated into the animal feed. This appears to be a convenient method to provide the animal feed. In particular, the ABM mycelium is grown on the grain substrate until the amount of mycelium is at least 10% (w/w) on dry weight of the mixture of grain and mycelium. Below this level, a relative high amount of the grain substrate needs to be mixed with other nutritional components in order to provide for an adequate economic effect. It is preferred that the ABM mycelium is grown on the grain substrate until the amount of mycelium is between 10 and 20% (w/w) on dry weight of the mixture of grain and mycelium.

In another embodiment, the one or more acids are chosen from C₁-C₁₆ aliphatic acids, in particular, C₁-C₇ small chain acids and/or C₈-C₁₆ medium chain acids.

In still another embodiment, the animal feed is nutritionally incomplete, for example, since the animal feed is provided as a so-called premix (to be mixed with other feed material). Thus, in order to produce a more complete animal feed, the nutritional incomplete animal feed has to be mixed with one or more other nutritional components such as, for example, proteins and/or carbohydrates and/or fats.

The disclosure further pertains to the use of the composition of the disclosure against resistant bacteria of the Enterobacteriaceae, in particular, of Salmonella and/or Escherichia. With the term “resistant bacteria” is meant bacteria that are resistant to conventional antibiotics. Examples of such resistant bacteria include cefotaxime-resistant Escherichia coli, carbapenem-resistant Enterobacteriaceae and extended spectrum beta lactamase-producing Escherichia coli (ESBL-producing E. coli). Preferably, the disclosure pertains to the use of the inventive composition in animal feed against resistant bacteria of the Enterobacteriaceae, in particular, of Salmonella and/or Escherichia.

### Examples

Example 1 describes an in vitro model study for assessing the effect of an antimicrobial on bacterial growth.

Example 2 describes an in vivo study for assessing the effect of ABM mycelium combined with organic acids on bacterial shedding.

Example 3 describes a second in vivo study for assessing the effect of ABM mycelium combined with organic acids on bacterial shedding.

Example 4 describes an in vivo study for assessing the effect of ABM mycelium combined with organic acids on bacterial shedding.

Example 5 describes an in vivo study with broilers assessing the transmission of Salmonella.

### BRIEF DESCRIPTION OF THE DRAWINGS

FIG. 1 shows the effect of ABM mycelium combined with organic acids on the shedding of Salmonella.

FIG. 2 shows the effect of ABM mycelium combined with organic acids on diarrhea.

FIG. 3 shows the effect of ABM mycelium combined with organic acids on the feed intake.

FIG. 4 shows the effect of ABM mycelium combined with organic acids on the feed efficacy.

FIG. 5 shows the effect of ABM mycelium combined with organic acids on the shedding of enterobacteriaceae in further in vivo studies.

### DETAILED DESCRIPTION

### Example 1

Example 1 describes an in vitro model study for assessing the effect of ABM mycelium on bacterial adhesion. In this method the adhesion of Salmonella typhimurium to ABM mycelium is assessed.

Use was made of a 96-well plate on which the ABM mycelium was coated. For this, the ABM mycelium (in this and each case below a fermented rye product was actually used, in which product the amount of ABM mycelium was about 15% w/w) was suspended in PBS to a final concentration of 1% (w/v) and mixed thoroughly. Subsequently the suspension was centrifuged to remove insoluble material. Thereafter, the supernatant was used for coating the wells of the microtiter plate. For the adhesion assessment, a Salmonella typhimurium suspension was added to the microtiter plate. The plate was then incubated for 30 minutes and after this incubation step washed with PBS. Subsequently growth medium was added to the wells and the time to onset OD600 value was determined. The optical density (OD) measurement was used as a tool to compare numbers of adhered bacteria to the coated wells of the 96-well plate with different compounds. The initial cell density of adhered bacteria correlates with the time-dependent detection of the growth by optical density measurement. A shorter time to onset OD600 value represents more adhesion of bacteria to the substrate, and hence an expected higher decrease of in vivo growth.

The results for the test with Salmonella typhimurium showed that the average time to onset OD600 was 4.9 hours (f 0.3 hour) as compared to the control (only PBS), which had an average time to onset OD600 of 7.3 hours (±0.1 hour). About twenty other compounds that were suspected of having a potential effect an adhesion (compounds not indicated in this example) showed an average time to onset OD600 generally between 5 and 8.5 hours.

In a second in vitro study, the test was repeated, and additionally the effect on Salmonella enteritidis and E. coli was measured. Also, the amount of ABM mycelium was used in the full amount (see above; denoted “100%”), half of this amount (“50%”) and a quarter of this amount (“25%”). The results are indicated here beneath in Table 1.

TABLE 1
Effect of ABM mycelium in various amounts on
the adhesion of various enterobacteriaceae,
by measuring the time to onset OD600 in hours.

| Compound | S. typhimurium | S. enteritidis | E. coli |
|---|---|---|---|
| Control | 7.0 | 6.3 | 7.1 |
| ABM 100% | 6.0 | 5.5 | 5.7 |
| ABM 50% | 5.7 | 5.5 | 5.9 |
| ABM 25% | 5.7 | 5.5 | 6.4 |

From the model studies it appears that mycelium of ABM has a significant effect on the adhesion of various enterobacteriaceae. The effect appears to be independent of the type of bacterium despite the fact that, in particular, the Escherichia bacteria are of a completely different species than the Salmonella bacteria. The amount of ABM mycelium does not appear to be critical to obtain the adhesion effect as such.

### Example 2

Example 2 describes an in vivo study for assessing the effect of ABM mycelium combined with organic acids on bacterial shedding. In this study, it was assessed whether the effect seen in vitro (see Example 1) indeed corresponds to in vivo bacterial shedding. In particular, it was assessed whether by introducing ABM mycelium in the feed of the pigs, in this case combined with an organic acids blend, the shedding of viable bacteria could be reduced. As controls, a negative control using the regular feed was used, and as a positive control the same feed with added butyrate, a particular short chain fatty acid that is commercially used in poultry feed to reduce bacterial shedding. The organic acid blend was a regular C₁-C₁₆ organic acid blend containing a combination of formic and lactic acid, added at 4 liters per 100 kg.

A total of 24 Topi*Hypor boar piglets were used. Only healthy male animals that did not receive antibiotics and that were negative for Salmonella (determined by qualitative examination of the feces) were included in the study. Animals were identified by uniquely numbered ear tags. Animals were divided over three treatment groups (8 animals per group) by weight and litter.

Piglets were individually housed (0.8×1.6 m) directly after weaning (24 days of age+/−3 days) in pens containing tenderfoot slatted floors. The first 24 hours after weaning continuous light was provided, thereafter 16 hours light and 8 hours darkness. Piglets received feed and drinking water ad lib. The different treatments were administered in the feed during the total study period (from weaning until the end of the study) as indicated below in Table 2.

TABLE 2
Feed treatments

| | No. of | | |
|---|---|---|---|
| Treatment | animals | Additives | Inclusion level |
| Negative control | 8 | — | — |
| Butyrate | 8 | Butyrate + acid blend | 6 kg/ton + 4 L/ton |
| ABM mycelium | 8 | ABM + acid blend | 2 kg/ton + 4 L/ton |

After 10 days piglets were orally infected with Salmonella typhimurium (in BHI medium) given by a pre-inoculated feed matrix containing 1 ml 1*10⁹ cfu/ml. Oral infection was performed in this way during 7 consecutive days.

Fecal sampling was performed at day 1, 2, 3, 4, and 7 post Salmonella infection. Samples were stored at 4 degrees and analyzed the next day. Samples were diluted and homogenized in BPW containing novobiocin. Serial dilutions were made and plated onto selective chromogenic agar plates, and incubated o/n at 37° C. Typical Salmonella colonies were counted and the amount (cfu/gram) was calculated. Of each sample two presumptive Salmonella colonies were confirmed by qPCR for both Salmonella and Salmonella typhimurium. When no colonies were observed in the lowest dilution plates the samples were screened for Salmonella presence (qualitative) after pre-enrichment by the conventional MSRV/XLD method.

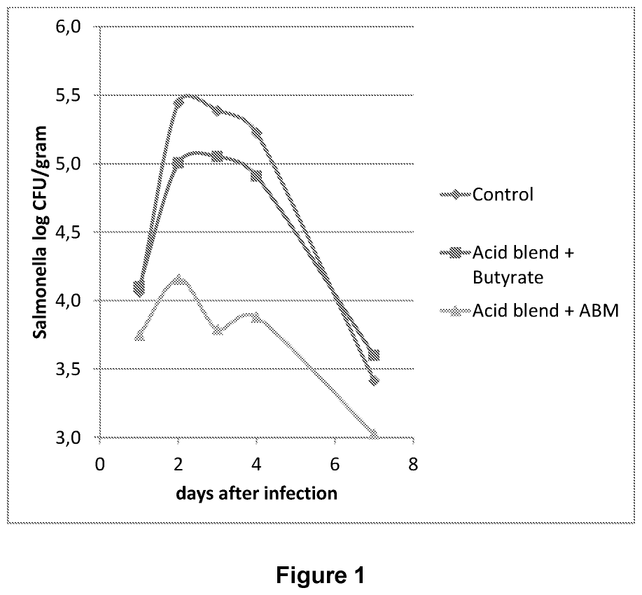

The results are indicated in FIG. 1, which shows the effect of ABM mycelium combined with organic acids on the shedding of Salmonella. It appears that the combination of mycelium of ABM and organic acids indeed has a significant effect on the shedding of viable salmonella bacteria. In particular, the effect is very large when compared to butyrate, a compound that is used in poultry for this purpose. It is thus also clear that the in vitro model (Example 1) is predictive for the in vivo reduction of bacterial shedding.

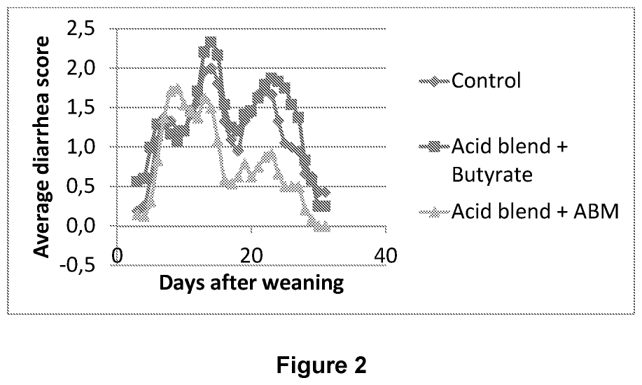

FIG. 2 shows the effect on diarrhea. A feces scoring was performed daily from day 3 after weaning until the end of the study. Diarrhea score was determined as: 0=normal feces; 1=flat feces; 2=wet feces; 3=watery feces. The results as depicted in FIG. 2 show a significant reduction of the ABM mycelium on diarrhea.

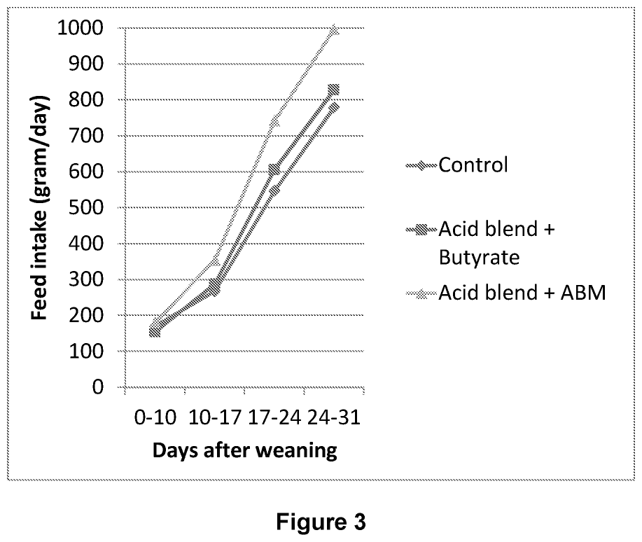

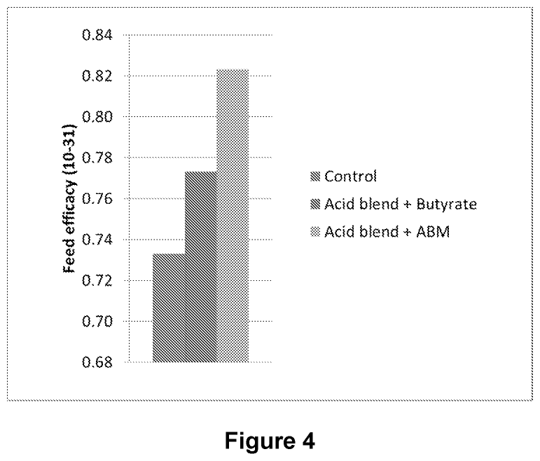

To assess performance, piglets were inspected daily. Body weight and feed intake were determined at weaning, before infection, and 7, 14, and 21 days after infection (day 0, 10, 17, 24, and 31). Feed efficacy was determined as gram growth/gram feed intake. FIG. 3 shows the effect of ABM mycelium combined with organic acids on the feed intake. FIG. 4 shows the effect of ABM mycelium combined with organic acids on the feed efficacy. The results show a significant positive impact on performance due to the presence of ABM mycelium in the feed.

### Example 3

Example 3 describes a second in vivo study for assessing the effect of ABM mycelium combined with organic acids on bacterial shedding. In this study, as a positive control the acid blend on itself was used (thus without the ABM mycelium.

A total of 36 Topi*Hypor boar piglets were used. Only healthy male animals that did not receive antibiotics and that were negative for Salmonella (determined by qualitative examination of the feces) were included in the study. Animals were identified by uniquely numbered ear tags. Animals were divided over three groups (12 animals per group) by weight and litter.

Piglets were individually housed (0.8×0.8 m) directly after weaning (24 days of age+/−3 days) in pens containing tenderfoot slatted floors. The first 24 hours after weaning continuous light was provided, thereafter 16 hours light and 8 hours darkness. Piglets received feed and drinking water ad lib. The different treatments were administered in the feed during the total study period (from weaning until the end of the study) as indicated below in Table 3.

TABLE 3
Feed treatments

| | No. of | | |
|---|---|---|---|
| Treatment | animals | Acid blend | ABM |
| Negative control | 12 | None | — |
| Acid blend | 12 | 4 L/ton | — |
| Acid blend + ABM mycelium | 12 | 4 L/ton | 2 kg/ton |

After 8 days, piglets were orally infected with Salmonella typhimurium (in BHI medium) given by a pre-inoculated feed matrix containing 1 ml 1*10⁹ cfu/ml. Oral infection was performed in this way during 7 consecutive days.

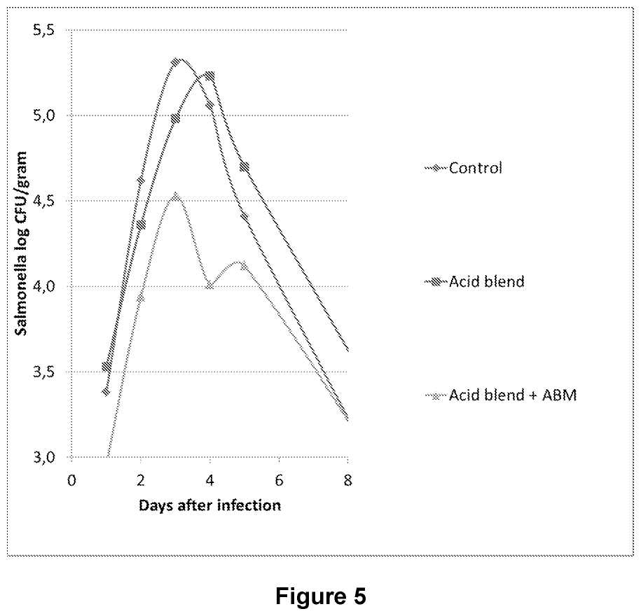

Fecal sampling was performed at day 1, 2, 3, 4, and 5 post Salmonella infection. Samples were stored at 4 degrees and analyzed the next day. Samples were diluted and homogenized in BPW containing novobiocin. Serial dilutions were made and plated onto selective chromogenic agar plates, and incubated o/n at 37° C. Typical Salmonella colonies were counted and the amount (cfu/gram) was calculated. Of each sample two presumptive Salmonella colonies were confirmed by qPCR for both Salmonella and Salmonella typhimurium. When no colonies were observed in the lowest dilution plates the samples were screened for Salmonella presence (qualitative) after pre-enrichment by the conventional MSRV/XLD method. The results are indicated in FIG. 5 and correspond to the results as indicated in FIG. 1.

The above in vivo experiment was repeated to assess the effect on Escherichia coli shedding by pigs. The experiment was run in correspondence with the salmonella experiment as described here above, with 10 animals being used per group. The results showed that on the day of artificial E. coli infection, none of the animals were positive in their feces for E. coli. At day 12, over 70% of the animals were positive in each group. Two days later, in the two control groups (negative control and acid blend group) the percentage of positive animals was 60%, whereas in the ABM group no shedders (0% of the animals tested positive for E. coli) were present at all.

### Example 4

An in vivo study was conducted according to the protocol described in Example 2, except that 12 animals per treatment group were used and the piglets were selected based on the presence of cefotaxime-resistant Escherichia coli; the selected animals were infected with Salmonella enteritidis after 5 days. In this study the shedding of cefotaxime-resistant Escherichia coli to ABM mycelium on rye and to a combination of ABM mycelium on rye and β-1,4-mannobiose at 50:50. The organic acid blend was a regular C₁-C₁₆ organic acid blend containing a combination of formic and lactic acid. The total amount of the agents is the same in all experiments.

The results for the test with cefotaxime-resistant Escherichia coli are shown in the Table below.

TABLE 4
Effect of ABM mycelium on rye and β-1,4-mannobiose
on the shedding of cefotaxime-resistant Escherichia
Coli, by measuring over the first 4 and over 16 days.

| | Amount | | |
|---|---|---|---|
| Compound | (kg/ton feed) | 1-4 days | 16 days |
| Control | — | 2.0 | 2.1 |
| Acid blend | 4 | 1.4 | 1.5 |
| ABM 100% | 2 | 0.8 | 1.0 |
| ABM 50%: β-1,4- | 2 | 0.5 | 0.9 |
| mannobiose 50% | | | |

The study shows that mycelium of ABM on rye with or without β-1,4-mannobiose have a significant effect on the adhesion of cefotaxime-resistant Escherichia coli. The acid blend alone does not provide a significant reduction of the adhesion of cefotaxime-resistant Escherichia coli.

### Example 5

An in vivo study was conducted using two groups, each group comprising 6 replicating pens with 30 birds. Three birds in each pen were infected with Salmonella enteritidis (seeder birds). The broilers were fed with a conventional broiler diet during 42 days. One group of broilers was treated with ABM mycelium on rye and an organic acid blend. The organic acid blend was a regular C₁-C₁₆ organic acid blend containing a combination of formic and lactic acid. The transmission of Salmonella to non-seeder birds was established by determining the number of infected or positive birds after 28 and 42 days.

After 28 days, the control (untreated) group consisted of 83% of infected birds, whereas the treated group contained 55% of infected birds. After 42 days, 60% of the birds were infected in the control group and 35% of positive birds in the treated group. This clearly demonstrates that the treatment aids in the containment of the Salmonella in the broilers.

1.-17. (canceled)

18. An animal feed comprising:
Agaricus blazei Murill (ABM) mycelium, and
one or more C₁-C₁₆ organic acids.

19. The animal feed of claim 18, wherein the ABM mycelium is present in the animal feed in an amount of 0.01 to 10 kg per metric ton of total daily intake of feed.

20. The animal feed of claim 19, wherein the ABM mycelium is present in the animal feed in an amount of 0.05 to 2 kg per metric ton of total daily intake of feed.

21. The animal feed of claim 18, wherein the one or more C₁-C₁₆ acids are present in the animal feed in an amount of 0.1 to 10 kg per metric ton of total daily intake of feed.

22. The animal feed of claim 21, wherein the one or more C₁-C₁₆ acids are present in the animal feed in an amount of 0.5 to 6 kg per metric ton of total daily intake of feed.

23. The animal feed of claim 18, wherein the ABM mycelium is grown on a grain substrate.

24. The animal feed of claim 23, wherein the grain substrate with the ABM mycelium grown thereon is incorporated into the animal feed.

25. The animal feed of claim 24, wherein the ABM mycelium is grown on the substrate until the amount of ABM mycelium is at least 10% (w/w) on a dry weight basis of the mixture of grain and mycelium.

26. The animal feed of claim 25, wherein the ABM mycelium is grown on the substrate until the amount of ABM mycelium is between 10 and 20% (w/w) of the total mass of the grain substrate.

27. The animal feed of claim 18, wherein the one or more C₁-C₁₆ organic acids are selected from C₁-C₁₆ aliphatic acids.

28. The animal feed of claim 18, wherein the one or more C₁-C₁₆ organic acids are selected from C₁-C₇ aliphatic acids.

29. The animal feed of claim 18, wherein the one or more C₁-C₁₆ organic acids are selected from C₈-C₁₆ aliphatic acids.

30. The animal feed of claim 18, wherein the animal feed is nutritionally incomplete.

31. The animal feed of claim 23, wherein the ABM mycelium is grown on a rye substrate.

32. The animal feed of claim 23, wherein the ABM mycelium is grown on a millet substrate.

33. A method of using the animal feed of claim 18, the method comprising:
feeding the animal feed to an animal.

34. A kit-of-parts comprising:
a first constituent part that comprises Agaricus blazei Murill (ABM) mycelium; and
a second constituent part comprising:
one or more C₁-C₁₆ organic acids, and, optionally,
an instruction to orally administer both the first and second constituent parts to an animal.

35. A method of producing an animal feed, the method comprising:
mixing Agaricus blazei Murill mycelium and one or more C₁-C₁₆ organic acids with protein(s) and/or carbohydrate(s) and/or fat(s) to produce the animal feed.
